(19) United States
(12) Patent Application Publication
(10) Pub. No.: US 2005/0119526 A1
(43) Pub. Date: Jun. 2, 2005

(54) Mouthpiece for endoscope

(75) Inventors: Jae-Seung Jeong, Anyang-city (KR); Soo Won Suh, Seoul (KR); Poong-Lyul Rhee, Seoul (KR)

Correspondence Address: Richard P. Berg;Ladas & Parry, Suite 2100, 5670 Wilshire Boulevard, Los Angeles, CA, 90036, US

(73) Assignee: TNO CO., LTD.

(21) Appl. No.: 10/998,314

(22) Filed: Nov. 26, 2004

(30) Foreign Application Priority Data
Nov. 28, 2003 (KR) 10-2003-0085631

(51) Int. Cl. A61B 1/00

(52) U.S. Cl. 600/114

(57) ABSTRACT

A mouthpiece for an endoscope includes a main body disposed to closely contact an outer side of a mouth and provided with an central insertion hole through which the endoscope is inserted, an guide body supported on the central insertion hole of the main body to guide insertion of the endoscope, and a movement preventing unit disposed in an oral cavity to prevent the mouthpiece from moving in the oral cavity. The guide body and the movement preventing unit may designed to be separable from the main body.

### BACKGROUND OF THE INVENTION

1. Field of the Invention

The present invention relates to a mouthpiece for an endoscope, and more particularly, to a mouthpiece that can be securely fixed on teeth of a patient to prevent the teeth from being damaged, allowing the endoscope to be smoothly inserted through thereof.

2. Description of the Related Art

Generally, an endoscope is a medical device for examining an internal human body to diagnose or cure a focus of a patient.

When the endoscope is used, since a doctor can examine the internal organs by eyes, the disease that may not be founded through an ultrasonography and an X-ray can be early found and treated.

The endoscope can be classified into an esophagus endoscope, a bronchial endoscope and a stomach endoscope, which are inserted into the internal body through a mouth and selectively used depending on a purpose and an examination region, and a breast endoscope and a abdominal endoscope, which are inserted into the internal body through an incised body, and a special endoscope that is used only for a special internal organ.

In order to use the esophagus, bronchial and stomach endoscopes that are inserted through the mouth, a mouthpiece has been used so that the endoscope can be smoothly inserted.

The mouthpiece includes a main body that is designed to be supported around a mouth of a patient and provided with a central insertion hole, a guide body formed on the central insertion hole to guide the insertion of the endoscope, and a tooth supporting body formed on a rear surface of the main body and fixed by front teeth of the patient to support the main body.

In use, when the patient fixes the tooth supporting body in a state where the main body contacts around the patient's opened mouth, the main body is supported on the mouth and the teeth to maintain the opened state of the patient's mouth.

In this state, when the endoscope is inserted through the central hole of the main body, the endoscope is inserted into the internal body of the patient via the mouth and the esophagus, while being guided by the guide body.

However, since the prior mouthpiece is designed to be supported by only the front teeth, the fixture of the mouthpiece may not be stable.

Accordingly, the patient may severely feel a pain or cough out the mouthpiece during the endoscopy. Furthermore, since the patient has to support the mouthpiece using his/her front teeth, the teeth may be possibly damaged.

In addition, since the guide body of the prior mouthpiece is formed in a linear-shape, it cannot fully depress a tongue that is curved in a predetermined curvature, thereby making it difficult for the endoscope to be smoothly inserted.

### SUMMARY OF THE INVENTION

Therefore, the present invention has been made in an effort to solve the above-described problems of the prior art.

Accordingly, it is an objective of the present invention to provide a mouthpiece for an endoscope that can be securely fixed on teeth, prevent the teeth from being damaged and allow the endoscope to be smoothly inserted into an internal body.

To achieve the above objective, the present invention provides a mouthpiece for an endoscope, comprising a main body disposed to closely contact an outer side of a mouth and provided with an central insertion hole through which the endoscope is inserted; and a movement preventing unit formed on a side of the main body, disposed in an oral cavity and fixed by front and grinding teeth to disperse fixing force applied to the teeth.

The movement preventing unit comprises a front support projected toward the inside of the oral cavity and a side support located on both sides of the front support and projected toward the inside of the oral cavity.

The front support and the side support are spaced away from each other by a predetermined gap.

At least one of the front and the side support is provided with a hook step preventing the mouthpiece from being removed out of the oral cavity.

The mouthpiece may further comprise a guide body extending from the central insertion hole toward an air way of a patient.

A rear end of the guide body is curved with a relatively high curvature to provide an enough passage space in the air way by depressing a rear end of a patient's tongue.

The guide body is provided with a longitudinal circumferential step to allow the endoscope to be smoothly inserted.

The movement preventing unit is designed to be separable from the main body.

The main body is provided with an extending portion contacting a portion of a patient chick, which corresponds to grinding teeth.

The side support is formed on the extending portion so that the side support can be fixed by the grinding teeth.

The front and/or side support is formed of material selected from the group consisting of silicon, polyurethane and natural rubber.

The guide body is formed of material selected from the group consisting of synthetic resin, metal and polymer.

In another aspect of the present invention, there is provided a mouthpiece for an endoscope, comprising a main body disposed to closely contact an outer side of a mouth and provided with an central insertion hole through which the endoscope is inserted; an guide body supported on the central insertion hole of the main body to guide insertion of the endoscope; and a movement preventing unit disposed in an oral cavity to prevent the mouthpiece from moving in the oral cavity, wherein the guide body and the movement preventing unit are designed to be separable from the main body.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a rear perspective view of a mouthpiece for an endoscope according to a preferred embodiment of the present invention;

FIG. 2 is a plane view of FIG. 1;

FIG. 3 is a side view of FIG. 1;

FIG. 4 is a rear view of FIG. 1; and

FIG. 5 is an explode perspective view of a mouthpiece for an endoscope according to another embodiment of the present invention.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

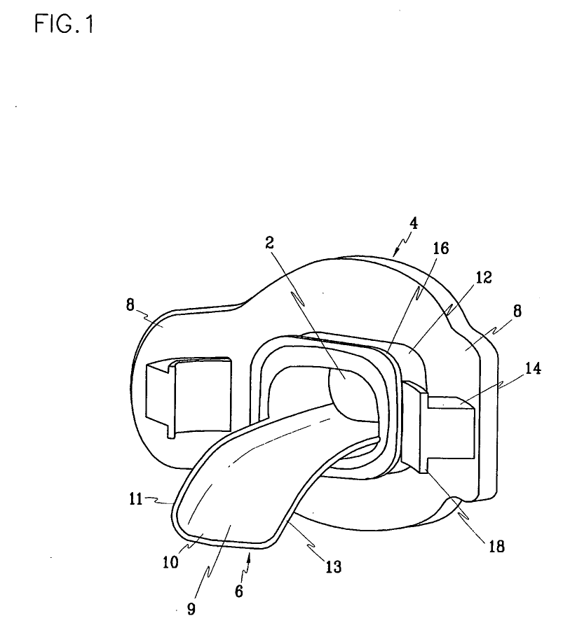

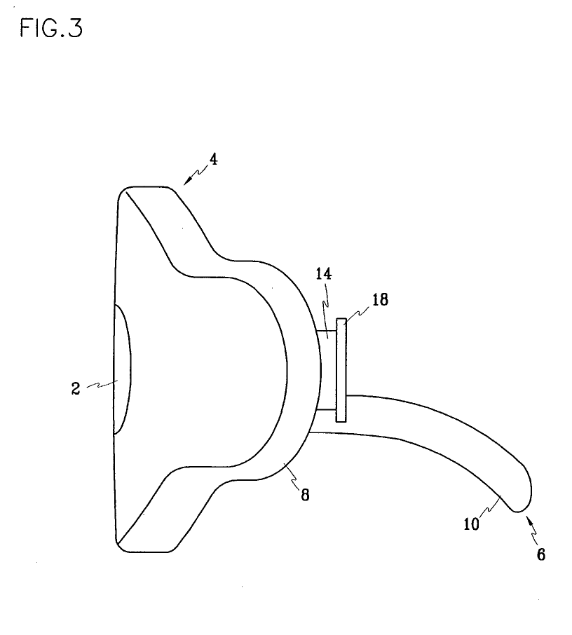

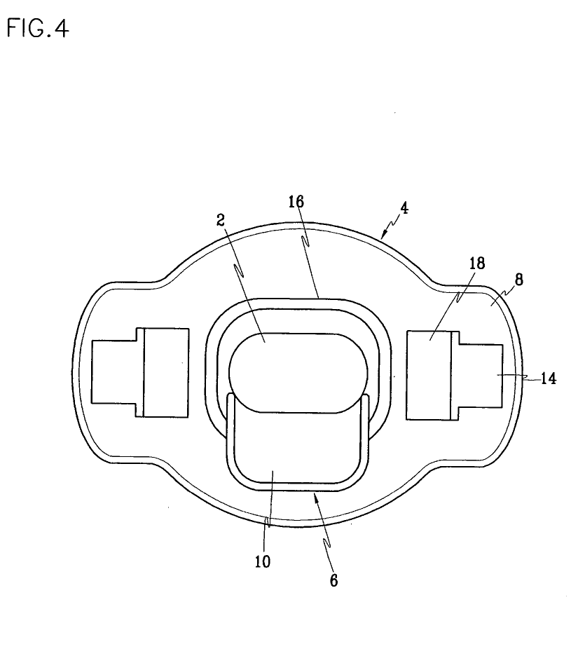

FIGS. 1 through 4 show a mouthpiece for an endoscope according to a preferred embodiment of the present invention.

As shown in the drawings, the inventive mouthpiece comprises a main body 4 that is designed to contact around a mouth and provided with a central insertion hole 2, a guide body 6 formed on the central insertion hole 2 to guide the insertion of an endoscope to an air way of a patient, and a movement preventing unit for preventing the main body 4 from being moving, the movement preventing unit being formed on a rear surface of the main body 4 to be located in the mouth.

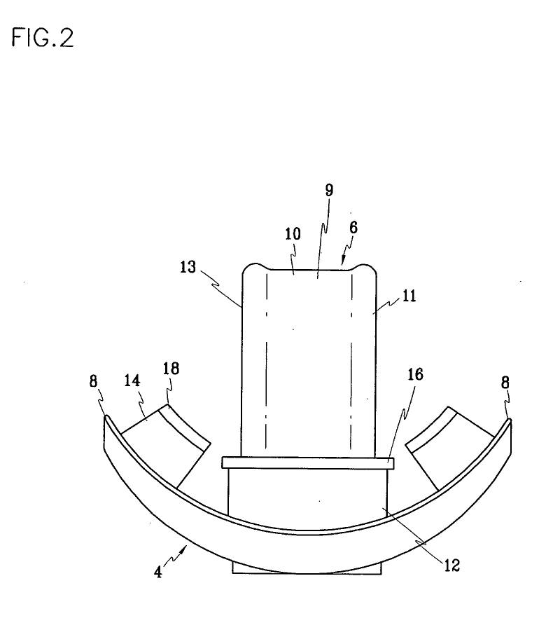

As shown in FIG. 2, the main body 4 is designed having a predetermined curvature and formed of elastic material so that it can closely contact around a lip. An extending portion 8 is formed on both ends of the main body 4 and extended to a portion of a cheek, which corresponds to grinding teeth.

The guide body 6 comprises a bottom guide portion 9 extending from the central insertion hole 2 to the air way and side guide portions 11 and 13 extending upward from both sides of the bottom guide portion 9. Therefore, the guide body 6 has a semi-cylindrical or a ⅓-⅙ cylindrical shape.

The guide body 6 is designed having a curvature similar or identical to that of a tongue so that the endoscope can be smoothly inserted. A distal end 10 of the guide body 6 is curved downward with the greater curvature so that it can depress a portion of the tongue adjacent to the air way, thereby securing the air way.

The movement preventing unit comprises a front support 12 and a side support 14 to securely support the mouthpiece in an oral cavity.

The front support 12 extends from an outer side of the central insertion hole 2 of the main body 4 toward the mouth so that it can be supported by the front teeth.

The front support 12 has a predetermined length so that it can be supported by the teeth. A hook step 16 is formed on a rear end of the front support 12 so that it cannot be easily removed from the mouth.

The side support 14 is projected from a rear surface of the extending portion 8 of the main body with a predetermined width and thickness so that it can be supported by the grinding teeth. The side support 14 is provided on both left and right sides of the main body 4.

The side support 14 is also provided at a rear end with a hook step 18 elevated to a predetermined height so that it cannot be easily removed from the grinding teeth.

As the front and side supports 12 and 14 are provided, the mouthpiece of the present invention can be simultaneously supported by the front and grinding teeth. As a result, the mouthpiece is not easily moved in the mouth. Particularly, since the mouthpiece is supported by both front and grinding teeth, the supporting force of the teeth is dispersed.

The front and side supports 12 and 14 may be formed of silicon, polyurethane, or natural rubber to prevent the teeth from being damaged. In order to securely depress the tongue, the guide body 6 may be formed of synthetic resin, metal or polymer that is harmless for the human body.

In use, the main body 4 contacts around the patient's mouth in a state where the mouth is opened. At this point, the front support 12 is fixed by the front teeth and the side support 14 is fixed by the grinding teeth. As a result, the main body 4 is supported by the mouth and teeth, maintaining the mouth opened.

In this state, the outer surfaces of the teeth contact the rear surface of the main body 4 and the extending portion 8. The inner surfaces of the teeth is supported on the hook steps 16 and 18 respectively formed on the front and side supports 12 and 14. As a result, the mouthpiece is securely supported in the oral cavity of the patient.

At this point, since the guide body 6 is curved with a predetermined curvature similar to that of the tongue, it suppresses the movement of the tongue. Furthermore, the rear end of the guide body 6 is curved toward the air way to depress the rear end of the tongue, thereby providing the sufficient air way passage through which the endoscope can be inserted.

In this state, the endoscope is inserted through the central insertion hole 2 of the main body 2, the endoscope is inserted into the internal body via the oral cavity and the esophagus while being guided by the guide body.

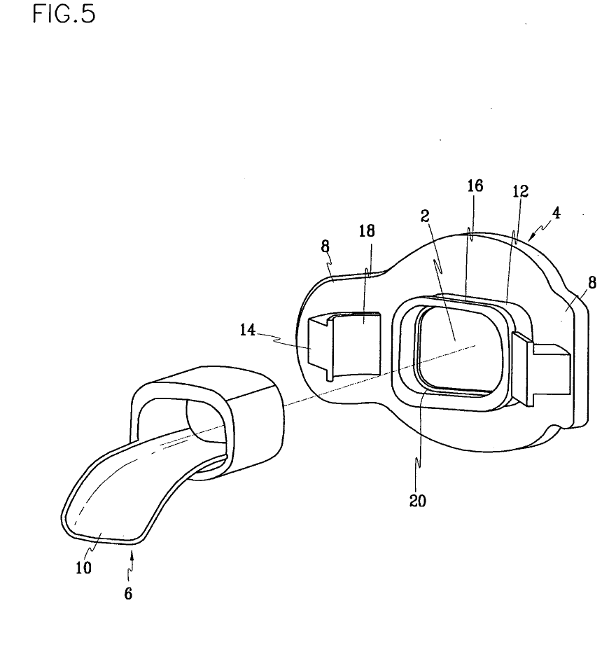

In the above embodiment, although the main body 4, the guide body 6 and the movement preventing unit are integrally formed in a single unit, the present invention is not limited to this. That is, as shown in FIG. 5, the front support 12 of the movement preventing unit and the guide body 6 may be designed to be disassembled from the main body 4. In this case, a hook step 20 is formed on the central insertion hole 2 of the main body and the guide body 6 and the front support 12 are integrally formed. A front end of the guide body 6 is inserted into the central insertion hole 2.

By doing this, a user can select a proper guide body and a movement preventing unit according to a mouth size of the patient. In addition, the user can select one from a variety of front supports 12 that are formed of different material according to a tooth condition of the patient.

According to the mouthpiece for the endoscope of the present invention, since the movement preventing unit is comprised of the front and side supports each having the elevated hook step, the mouthpiece can be securely fixed in the patient's mouth and dispersing the force applied to the patient's teeth.

Furthermore, since the front support is designed to be separable from the main body, the user can select one from a variety of front supports 12 that are formed of different material and in a different size according to a tooth condition of the patient.

In addition, since the guide body is formed having a curvature similar or identical to that of a patient's tongue, the patient can feel the comfort when his/her tongue is depressed by the guide body.

It will be apparent to those skilled in the art that various modifications and variations can be made in the present invention. Thus, it is intended that the present invention covers the modifications and variations of this invention provided they come within the scope of the appended claims and their equivalents.

1. A mouthpiece for an endoscope, comprising:
a main body disposed to closely contact an outer side of a mouth and provided with an central insertion hole through which the endoscope is inserted; and
a movement preventing unit formed on a side of the main body, disposed in an oral cavity and fixed by front and grinding teeth to disperse fixing force applied to the teeth.

2. The mouthpiece of claim 1, wherein the movement preventing unit comprises a front support projected toward the inside of the oral cavity and a side support located on both sides of the front support and projected toward the inside of the oral cavity.

3. The mouthpiece of claim 2, wherein the front support and the side support are spaced away from each other by a predetermined gap.

4. The mouthpiece of claim 2, wherein at least one of the front and side supports is provided with a hook step preventing the mouthpiece from be removed out of the oral cavity.

5. The mouthpiece of claim 1, further comprising an guide body extending from the central insertion hole toward an air way of a patient.

6. The mouthpiece of claim 5, wherein an rear end of the guide body is curved with a relatively high curvature to provide an enough passage space in the air way by depressing a rear end of a patient's tongue.

7. The mouthpiece of claim 5, wherein the guide body is provided with a longitudinal circumferential step to allow the endoscope to be smoothly inserted.

8. The mouthpiece of claim 1, wherein the movement preventing unit is designed to be separable from the main body.

9. The mouthpiece of claim 1, wherein the main body is provided with an extending portion contacting a portion of a patient chick, which corresponds to grinding teeth.

10. The mouthpiece of claim 9, wherein the side support is formed on the extending portion so that the side support can be fixed by the grinding teeth.

11. The mouthpiece of claim 2, wherein the front and/or side support is formed of material selected from the group consisting of silicon, polyurethane and natural rubber.

12. The mouthpiece of claim 5, wherein the guide body is formed of material selected from the group consisting of synthetic resin, metal and polymer.

13. A mouthpiece for an endoscope, comprising:
a main body disposed to closely contact an outer side of a mouth and provided with an central insertion hole through which the endoscope is inserted;
an guide body supported on the central insertion hole of the main body to guide insertion of the endoscope; and
a movement preventing unit disposed in an oral cavity to prevent the mouthpiece from moving in the oral cavity,
wherein the guide body and the movement preventing unit are designed to be separable from the main body.

14. The mouthpiece of claim 13, wherein the movement preventing unit comprises a front support projected toward the inside of the oral cavity and a side support located on both sides of the front support and projected toward the inside of the oral cavity.

15. The mouthpiece of claim 14, wherein at least one of the front and side supports is provided with a hook step preventing the mouthpiece from be removed out of the oral cavity.

16. The mouthpiece of claim 15, wherein an rear end of the guide body is curved with a relatively high curvature to provide an enough passage space in the air way by depressing a rear end of a patient's tongue.

17. The mouthpiece of claim 14, wherein the front and/or side support is formed of material selected from the group consisting of silicon, polyurethane and natural rubber.

18. The mouthpiece of claim 13, wherein the guide body is formed of material selected from the group consisting of synthetic resin, metal and polymer.
